(19) United States
(12) Patent Application Publication
(10) Pub. No.: US 2007/0104730 A1
(43) Pub. Date: May 10, 2007

(54) Methods and compositions employing pomegranate extracts and fementation products of statin-producing fungi

(75) Inventors: Peilin Guo, Gardena, CA (US); Jarrow Rogovin, Los Angeles, CA (US); Ephraim Lansky, Haifa (IL)

Correspondence Address: DR. MARK FRIEDMAN LTD.;C/o Bill Polkinghorn, 9003 Florin Way, Upper Marlboro, MD, 20772, US

(73) Assignees: Jarrow Formulas; Rimonest Ltd.

(21) Appl. No.: 11/270,564

(22) Filed: Nov. 10, 2005

(51) Int. Cl. A61K 36/06; A61K 36/185

(52) U.S. Cl. 424/195.16; 424/777

(57) ABSTRACT

Dietary supplements useful for treating or preventing cardiac disease are disclosed According to some embodiments, the presently disclosed supplement includes a pomegranate product fermented with a statin-producing fungus such one or more of Monascus, Aspergillus, Penicillium, Pleurotus, Pythium, Hypomyces, Paelicilomyces, Eupenicillium, and Doratomyces. Alternatively or additionally, the presently disclosed supplement includes a pomegranate product in combination with a plant product, such as a grain product and a legume, fermented with the statin-producing fungus. Optionally, the supplement includes at least one of coenzymeQ10 and a tocopherol. The presently disclosed dietary supplements include both a COX-2 inhibitor and a HMG-CoA reductase inhibitor, and in some embodiments, the supplement is useful for reducing or controlling blood cholesterol for managing atherosclerotic disease, and/or for managing blood pressure. Methods of preparing the presently disclosed dietary supplement are also provided.

### FIELD OF THE INVENTION

The present invention relates to dietary supplements suitable for treating or preventing cardiac disease including pomegranate extracts and fermentation products of statin-producing fungi.

### BACKGROUND OF THE INVENTION

Cardiovascular disease is a leading cause of morbidity and mortality, particularly in the United States and in Western European countries and is emerging in developing countries. Several factors are mentioned relation to the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesteremia, contribute to the development of atherosclerosis, a primary cause of vascular and heart disease.

Atherosclerosis is a disease characterized by the deposition of fatty substances, primarily cholesterol, and subsequent fibrosis in the inner layer (intima) of an artery, resulting in plaque deposition on the inner surface of the arterial wall and degenerative changes within it. The ubiquitous arterial fatty plaque is the earliest lesion of atherosclerosis and is a grossly flat, lipid-rich atheroma consisting of macrophages (white blood cells) and smooth muscle fibers. The fibrous plaque of the various forms of advanced atherosclerosis has increased intimal smooth muscle cells surrounded by a connective tissue matrix and variable amounts of intracellular and extracellular lipid. At the luminal surface of the artery, a dense fibrous cap of smooth muscle or connective tissue usually covers this plaque or lesion. Beneath the fibrous cap, the lesions are highly cellular consisting of macrophages, other leukocytes and smooth muscle cells. Deep in this cell-rich region may be areas of cholesterol crystals, necrotic debris and calcification.

If allowed to progress, the disease can cause narrowing and obstruction of the lumen of the artery, diminished or occluded blood flow and, consequently, ischemia or infarction of the predominantly affected organ or anatomical part such as the brain, heart, intestine or extremities. The result can be significant loss of function, loss of cellular substance, emergency medical and/or surgical procedures, and significant disability or death. Alternatively, the arterial wall can be severely weakened by the infiltration of tie muscular layer with the lipid (cholesterol), inflammatory white blood cells, connective tissue and calcium, resulting in soft and/or brittle areas which can become segmentally dilated (aneurysmal) and rupture or crack leading to organ, limb or even life-threatening hemorrhage.

Elevated low-density lipoprotein cholesterol (hereafter “LDL-cholesterol”) is directly related to an increased risk of coronary heart disease.

Statins and Statin-Producing Fungi

Statins are compounds that are known to have a lowering effect on levels of LDL-cholesterol in the human blood. Statins inhibit the hydroxymethylglutaryl coenzyme A (HMG-CoA) reductase, the rate-determining step in the cholesterol biosynthesis.

Scientific research has confirmed the healthy properties of statins especially with respect to LDL blood-cholesterol and triglyceride levels lowering activities, both in animals and in humans (Li et al., Nutrition Research 18, 71-81 (1998); Heber et al., Am. J. Clin. Nutr. 69, 231-236 (1999)).

Early reports of the effect of statins were made in 1979. The Japanese scientist Endo isolated a metabolite from Monascus that reduced artificially induced hyperlipoproteinemia in rats (Endo, J. Antibiotics 32, 852-854, (1979)). These metabolites are known as monacolins. Monacolin is identical to the cholesterol lowering pharmaceutical lovastatin. Lovastatin is sold by Merck co. under the tradename Mevacor. A derivative of lovastatin, simvastatin, is sold as a cholesterol-lowering drug under the name of Zocor. Other derivatives of lovastatin e.g. pravastatin, and mevastatin, are also sold as lipid lowering drugs against hypercholesterolemia. Monascus-extracts are sold in capsules in Japan as the dietary product Monacolin. The usual dose of the above statins is 20 mg/day, which results in at least 20% blood LDL-cholesterol lowering.

Nevertheless, many consumers seek natural alternatives to synthetic pharmaceutical products to aid with a variety of ailments experienced during daily life. Thus, dietary food supplements containing natural substances such as St. Johns wort, Gingko biloba, ginseng, and others have recently been marketed for a variety of purposes. There is an ongoing need for dietary supplements for treating or preventing cardiac disease containing natural substances.

As a food product, rice fermented with a red Monascus fungus (red rice) has been known and used for hundreds of years in China. Red rice was used and still is used in wine making, as a food-coloring agent and as drug in traditional Chinese medicine. Red rice powder capsules are sold as dietary supplements under the name of Cholestin by the firm Pharmanex. Pharmanex also sells a Cholestin bar containing red yeast rice (Monascus purperus went).

The production of statins is also reported in fermentation using fungi other than the above-mentioned Monascus species. It has been shown that statins can be produced by a variety of filamentous fungi, including Monascus, Aspergillus, Penicillium, Pleurotus, Pythium, Hypomyces, Paelicilomyces, Eupenicillium, and Doratomyces.

A “statin-producing” fungus is one which can be fermented to produce a product having a statin content of at least 0.05%, preferably at least 2%.

The red rice product is the fermentation product of at least one of the following Monascus fungi set forth in the table below

Red rice is the fermentation product of one or a mixture of Monascus fungi, comprising chiefly Monascus purperus Went, and in lesser proportions other Monascus species, e.g., Monascus ruber van Tieghem, Monascus Fuliginosus Sato, Monascus Pilosus Sato and Monascus albidus Sato. Red rice can also be the fermentation product of the following strains of Monascus including but not limited to Monascus albidus Sato, Monascus pilosus Sato, Monascus pubigerus Sato, Monascus ruber van Tieghem, Monascus paxii Lingelsheim, Monascus fuliginosus Sato, and Monascus purperus Went.

Recently, the blood pressure-lowering effect of statins were studied (see Terzoli et al., “Lowering of elevated ambulatory blood pressure by HMG-CoA reductase inhibitors,” J Cardiovasc Pharmacol. September 2005;46(3):310-5 and Kanbay et al. “Statin therapy helps to control blood pressure levels in hypertensive dyslipidemic patient,” Ren Fail. 2005;27(3):297-303). It is noted statins were found to moderately but significantly lower blood pressure in patients with high ambulatory blood pressure (ABP)

Pomegranate Extracts

Pomegranate (Punica granatum) has long been recognized as a fruit with many benefits for health¹. The plant is botanically unique, having actually only one true botanical relative, the pomegranate precursor, Punica protopunica, restricted to tile isolated island Socotra off the coast of Yemen. Corresponding to this botanical uniqueness is a parallel distinctiveness in terms of biochemistry. For example, pomegranate has long been recognized as the richest plant source of the female steroid hormone estrone², and recently, the male hormone testosterone and another female steroid, estriol, have also been discovered in pomegranate seed oil.³ A wide range of polyphienolic compounds including flavonoids, anthocyanins and tannins have been characterized both in pomegranate juice.⁴ and pericarp.⁵ Further, concentrations of these polyphenols extracted both from the fermented juice and the oil have been shown to be potently antioxidant in vitro and to additionally inhibit the eicosanoid enzyme lipoxygenase, and in the case of the polyphenols extracted from pomegranate seed oil, to also be significantly inhibitory of another eicosanoid pathway enzyme, cyclooxygenase (COX).
¹Frawley, D and Lad, V. The Yoga of Herbs: An Ayurvedic Guide to Herbal Medicine, Lotus Press, Twin Lakes, Wis. 1986

²Moneam, N. M. A., El Sharaky, A. S., and Badreldin, M. M. Oestrogen content of pomegranate seeds. Journal of Chromotography 438: 438-442, 1988

³Abd El Wahab, S. M., El Fiki, S. F., Mostafa, S. F. and Hassan, A. E. B, Characterization of certain steroid hormones in Punica granatum L. seeds. Bulletin of the Faculty of Pharmacy of Cairo University 36(1): 11-15, 1998.

⁴Artik, N., Cemeroglu, B., Burakami, H., and Mori, T. Determination of phenolic compounds in pomegranate juice by HPLC. Fruit Process 8 (12): 492-499, 1998.

⁵Ben Nasr, C., Ayed, N., and Metche, M. Quantitative determination of the polyphenolic content of pomegranate peel. Z Lebensm Unters Forsch 203 (4): 374-378, 1996.

U.S. Pat. No. 6,641,850 discloses usage of pomegranate extracts to treat atherosclerosis and to decrease the incidence of stroke or heart attack.

It is noted that both COX inhibitors (for example, rofecoxib, and celecoxib, marketed as VIOXX and CELEBREX by Merck and Searle/Pfizer respectively) and statin compositions (for example, lovastatin, marketed under the trademark MEVACOR by Merck, and described, among other places in U.S. Pat. No. 4,231,938; simvastatin, marketed under the trademark ZOCOR by Merck, and described, among other places in U.S. Pat. No. 4,444,784; pravastatin, marketed under the trademark PRAVACOL by Bristol-Myers-Squibb, and described, among other places, in U.S. Pat. No. 4,346,227; atorvastatin calcium, marketed under the name LIPITOR by Parke-Davis) are readily available as synthetic drugs. Nevertheless, as noted above, many consumers prefer natural substances to synthetic drugs, Therefore, it is clear that there is a need for a natural and pharmacologically acceptable composition for treating or preventing cardiac disease. In particular, there is an ongoing medical need for natural and pharmacologically acceptable compositions for reducing or controlling blood cholesterol, managing atherosclerotic disease, and for managing blood pressure.

The following patents and published non-patent references provide potentially relevant background material, and each publication is incorporated by reference in its entirety:
U.S. Pat. No. 6,849,281; U.S. Pat. No. 6,632,428; U.S. Pat. No. 6,576,242; U.S. Pat. No. 6,849,281; U.S. Pat. No. 6,544,525; U.S. Pat. No. 6,541,006; U.S. Pat. No. 6,541,005; U.S. Pat. No. 6,436,406; U.S. Pat. No. 6,641,850; U.S. Pat. No. 6,046,022; U.S. Pat. No. 6,534,540; US 2005/0147620; US 2003/0194413; US 2003/0133920; US 2003/0108657; US 2003/0104004; US 2005/011312 of one of the present inventors; US 2002/01341 of one of the present inventors; and US 2002/012710 of one of the present inventors

### SUMMARY OF THE INVENTION

The aforementioned needs are satisfied by several aspects of the present invention.

It is now disclosed for the first time a dietary supplement useful for treating or preventing cardiac disease comprising a pomegranate product, and a plant product fermented with a statin producing fungus, for example, a fungus from the Monascus genus.

Not wishing to be bound by theory, it is noted that the dietary supplements of the present inventor include both a COX-2 inhibitor and a HMG-CoA reductase inhibitor. Both COX-2 inhibitors and HMG-CoA inhibitors have been identified in the literature as effective for promoting cardiovascular health. The present invention provides a natural dieatary supplement comprising a combined product where there is a supra-additive synergistic effect.

According to some embodiments, the presently disclosed supplement comprises between about 5% to 95% wt/wt pomegranate product in combination with about 5% to 95% wt/wt fermented plant product. Preferably, the presently disclosed supplement comprises between about 70% wt/wt pomegranate product in combination with about 30% wt/wt fermented plant product.

According to some examples, both the pomegranate product and the fermented plant product are provided as a powder, which may be mixed to form the dietary supplement.

According to some embodiments, the plant product includes a plant seed product such as a grain product, e.g. a fermented grain product. Appropriate grains include but are not limited to barley, wheat, rice, corn, oats, spelt, buckwheat and rye.

Alternatively or additionally, the plant product includes a legume such as soybeans.

Alternatively or additionally, the plant product includes a ground up peel of a fruit, which, like seed products, may provide nutrients for the fungus.

Optionally, the supplement includes at least one of coenzyme Q₁₀ (for example, at a concentration between 0.1% and 10% wt/wt) and a tocopherol (for example, at a concentration between 0.1 and 5% wt/wt).

It is now disclosed for the first time a dietary supplement useful for treating or preventing cardiac disease comprising a pomegranate product fermented with a statin producing fungus, for example, a fungus from the Monascus genus.

In some embodiments, the pomegranate powder is provided as a pre-flowing powder, which when combined with powdered fungus, mixed, and fermented produces a compound useful in a dietary supplement. In some embodiments, 0.1-100 mg of fungus powder per gram of pomegranate powder is mixed with the pomegranate powder and fermented. Preferably, 1-10 mg of fungus powder per gram of pomegranate powder is mixed with the pomegranate powder and fermented.

According to some embodiments, the pomegranate product includes a pomegranate seed component. Exemplary pomegranate seed components include but are not limited to seed cakes, seeds, milled seeds and seed powder.

According to some embodiments, the supplement further comprises a plant product other than said pomegranate product (for example, a grain product such as rice) fermented with a said statin-producing fungus. Thus, in some examples, more the supplement includes more than one plant product fermented with the statin-producing fungus, i.e. the pomegranate product and another plant product. Not wishing to be bound by theory, it is disclosed that this can induce a synergistic effect.

Although any pomegranate product fermented with a statin producing fungus is within the scope of the present invention, it is noted that in some embodiments, a pomegranate seed cake is fermented with the statin producing fungus. According to some embodiments, the fermented seed cake is dried into a powder, which is combined with a pomegranate juice component to form a slurry, which is subsequently dried to form the powder.

According to some embodiments, the presently disclosed supplement includes a pomegranate product (for example, pomegranate seed cake) fermented with the statin producing fungus in combination with another plant product (for example, a grain product such as rice) fermented with the statin producing fungus.

According to some embodiments, the pomegranate product includes a pomegranate seed component, and the pomegranate seed is at least partially oil extracted.

According to some embodiments, the pomegranate product includes a fruit component.

It is now disclosed for the first time an article of manufacture comprising any of the aforementioned dietary supplements, packaging material and instructions for use identifying product as useful for a least one of managing a blood cholesterol level, managing a blood triglyceride level, reducing systolic blood pressure, reducing serum angiotensin converting enzyme (ACE) activity and managing atherosclerotic disease.

It is now disclosed for the first time an article of manufacture comprising any of the aforementioned dietary supplements, packaging material and instructions for use wherein the product is supplied in an orally administrable form selected from the group consisting of consisting of a tablet and a capsule.

It is now disclosed for the first time a method for lowering blood pressure in a hypertensive patient in a subject, the method comprising administering to a patient an oral formulation comprising a physiologically effective amount of a pomegranate product and a plant product fermented with a statin producing fungus, for example, a fungus from the Monascus genus.

It is now disclosed for the first time a method for correcting or preventing a blood lipid dyscrasia in a subject, the method comprising administering to a patient an oral formulation comprising a physiologically effective amount of a pomegranate product and a plant product fermented with a statin producing fungus, for example, a fungus from the Monascus genus.

It is now disclosed for the first time a method for treating or preventing cardiac disease of a subject, the method comprising administering to a patient an oral formulation comprising a physiologically effective amount of a pomegranate product and a plant product fermented with a statin producing fungus, for example, a fungus from the Monascus genus.

It is now disclosed for the first time a method for correcting or preventing a blood lipid dyscrasia in a subject, the method comprising administering to a patient an oral formulation comprising a physiologically effective amount of a composition comprising a pomegranate product fermented with a statin-producing fungus, for example, a fungus from the Monascus genus.

It is now disclosed for the first time a method for lowering blood pressure in a hypertensive patient in a subject, the method comprising administering to a patient an oral formulation comprising a physiologically effective amount of a composition comprising a pomegranate product fermented with a statin-producing fungus for example, a fungus from the Monascus genus

It is now disclosed for the first time a method for treating or preventing cardiac disease in a subject, the method comprising administering to a patient an oral formulation comprising a physiologically effective amount of a composition comprising a pomegranate product fermented with a statin-producing fungus, for example, a fungus from the Monascus genus.

It is now disclosed for the first time an article of manufacture comprising any of the aforementioned dietary supplements, packaging material and instructions for use wherein the product is supplied in an orally administrable form selected from the group consisting of consisting of a tablet and a capsule.

According to some embodiments, cardiac disease is treated or prevented upon consumption of the one to three tablets or capsules per day of any of the aforementioned dietary supplements, where each tablet is between 200 mg and 500 mg per 70 kg of patient In some embodiments, the tablet or capsule is administered for at least 6 weeks of time.

Optionally, the oral formulation includes an acceptable pharmaceutical carrier.

According to some embodiments, the oral administration is employed to manage a blood cholesterol level.

According to some embodiments, the oral administration is employed to manage a level of blood triglycerides.

According to some embodiments, the oral administration is employed to manage atherosclerotic disease.

According to some embodiments, the oral formulation includes at least one of coenzymeQ₁₀ and a tocopherol.

It is now disclosed for the first time a method of preparing a dietary supplement useful for treating or preventing cardiac disease. The presently disclosed method includes the steps of providing a pomegranate product and fermenting the pomegranate product with a statin-producing fungus.

According to some embodiments, step of fermenting the pomegranate product includes fermenting a pomegranate seed product such as seed cake. In some embodiments, the fermented pomegranate seed product is optionally dried, and combined with a pomegranate fruit product In some embodiments, this combined product is dried to form the dietary supplement.

According to some embodiments, the pomegranate product is provided as a powder which is mixed with the fungus (e.g. a fungus powder). The mixture is subsequently fermented.

Optionally, the method includes the step adding to the fermented pomegranate product at least one of coenzymeQ₁₀ and a tocopherol.

It is now disclosed for the first time a method of preparing a dietary supplement useful for treating or preventing cardiac disease. The presently disclosed method includes the steps of providing a pomegranate product, providing a plant product fermented with a statin-producing fungus, and mixing the pomegranate product and the plant product to form the dietary supplement.

It is noted that throughout this disclosure, appropriate statin-producing fungi include but are not limited to of Monascus, Aspergillus, Penicillium, Pleurotus, Pythium, Hypomyces, Paelicilomyces, Eupenicillium, and Doratomyces.

Appropriate varieties of Monascus fungus include but are not limited to Monascus purperus, Monascus ruber, Monascus fuliginosus, Monascus pilosus, and Monascus albidus.

These and further embodiments will be apparent from the detailed description and examples that follow.

### DETAILED DESCRIPTION OF THE INVENTION

Pomegranate (Punica granatum) extracts have been extensively studied and found to be highly efficacious in preventing or even reversing atherosclerosis (for example, see Aviram and Dornfeld, Atherosclerosis. September 2001;158(1):195-8). Relevant mechanisms include inhibiting the uptake of cholesterol by macrophages (foam cells), inhibiting oxidation of LDL, and other mechanisms including a modest inhibition of serum cholesterol production. In particular, concentrations of polyphenols extracted both from fermented pomegranate juice and pomegranate oil have been shown to be significantly inhibitory of cyclooxygenase (COX), which facilitates transformation of a substance called squalene to cholesterol. Furthermore, flee flowing pomegranate powder, such as the powder disclosed in US 2005/0118312 of one of the present inventors, is also a source of COX inhibitors.

Earlier in the biochemical cycle that produces cholesterol is a substance called Acetyl-CoA enzyme It is converted to an intermediate called mevalonate by an enzyme called 3-hydroxy-3-methylglutamate-CoA reductase (“HMG-CoA”). Recent pharmaceutical advances have produced a number of substances that inhibit the activity of HMG-CoA and slow the production of cholesterol. HMG-CoA inhibitors have been used and are claimed to be used to reduce cholesterol to slow various blood vessel and related heart disease problems which we generally refer to as cardiovascular disease.

Red yeast rice (RYR), and soybeans fermented with red rice yeast (Monascus purperus) contain statins and are known to be effective inhibitors of the enzyme HMG Co-A reductase, and are effective in lowering serum cholesterol. Pomegranate is not known as a source of statin compounds.

In other applications (see, for example, U.S. Pat. No. 6,534,540 which is drawn to treatments of cancer), the combination of HMG Co-A Reductase and COX inhibitors (such as occur pomegranate powder) was more effective in inhibiting prostate cancer than either individually. It thus appears that compositions providing a combination of HMG Co-A Reductase and COX inhibitors would also be effective in suppressing atherosclerosis and lowering serum cholesterol. In particular, mixtures comprising a pomegranate product fermented with a Monascus fungi, and mixtures comprising a pomegranate product in combination with a plant product fermented with a Monascus funi are proposed for treating or preventing cardiac disease.

It is noted that administration of red yeast rice in combination with anti-oxidants such as coenzyme Q₁₀ and tocopherols is known to be effective for treating artherosclerosis and for reducing or controlling blood cholesterol and triglycerides (see, for example, U.S. Pat. No. 6,576,242). Furthermore, it is known that pomegranate juice possesses impressive antioxidative properties due to its high flavonoid content, mainly the water soluble tannins and proanthocyanidins⁶. In healthy humans, pomegranate juice consumption also demonstrated potent antioxidative capabilities against lipoprotein oxidation, and also increased PON1 activity and improved serum total antioxidant status. Thus, it is believed that pomegranate extract, known for its antioxidative properties, in combination with a plant product fermented with a statin producing fungus is similarly effective for treating artherosclerosis and for reducing or controlling blood cholesterol and triglycerides.
⁶ Gil M I, Tomas-Barberan F A, Hess-Pierce B, et al Antioxidant activity of pomegranate juice and its relationship with phenolic composition and processing. J Agric Food Chem 2000; 10 4581-89.

This notion is further reinforced by other results related to administration of HMG Co-A Reductase in combination with COX inhibitors. Thus, Winokur, PCT Appl. U.S. Ser. No. 98/21901, filed Oct. 16, 1998, published as W099/20110 entitled “Combination Therapy for Reducing the Risks Associated with Cardio and Cerebrovascular Disease”, and a corresponding U.S. Pat. No. 6,245,797, claims a combination of a COX-2 inhibitor with an HMG-CoA inhibitor for treating, preventing, and/or reducing the risk of atherosclerosis and atherosclerotic disease events and a method of using a COX-2 inhibitor with an HMG-CoA inhibitor for treating, preventing, and/or reducing the risk of atherosclerosis and atherosclerotic disease events. Another patent, Nichtberger, U.S. Pat. No. 6,136,804, Oct. 24, 2000, entitled “Combination therapy for treating, preventing, or reducing the risks associated with acute coronary ischemic syndrome and related conditions” proposes the utilization for an antiplatelet agent in combination with a therapeutically effective amount of a COX-2 inhibitor to treat, prevent or reduce the risk of acute coronary ischemic syndrome, thrombosis, and related vascular problems.

Optionally, the composition of the present invention includes one or more additional antioxidants (other than the anti-oxidants of pomegranate extracts) that neutralizes free radicals in the body. For example, Vitamin E, a potent antioxidant, has been shown to reduce the extent of atherosclerosis in several animal models and studies have shown that Vitamin E can be protective against the disease. Pryor 28(1) Free Radical Biology & Medicine 141-64 (2000). The development of the fatty streak lesion may be based upon two factors: the presence of elevated plasma LDL and its oxidative modification within the artery wall. LDL particles in whole plasma contain the antioxidant compounds vitamin E and β-carotenes and the plasma itself contains antioxidants that protect the LDL for a relatively short time. Under pro-oxidant conditions, the vitamin E and β-carotene are destroyed before the fatty acids undergo peroxidation. Id., at 921. It is likely that decreases in vitamin E and beta-carotene are early events reflecting the initial stages of lipid peroxidation. Witztum & Steinberg, 88(6) J. Clinical Investigation 1785-1792 (1991).

Another important antioxidant known in the art is Coenzyme Q₁₀. Coenzyme Q₁₀ (Ubiquinone) is a naturally occurring substance that plays a central role in oxidative respiration as a catalyst and has a separate direct membrane stabilizing effect. In man, vitamin E, beta-carotene, and Coenzyme Q₁₀ all appear to be endogenous antioxidants in LDL. Epidemiologic data suggest a negative correlation between coronary disease and levels of vitamin E. It is also an antioxidant and free radical scavenger, and protects ischemic tissue from the damage that occurs when blood flow is restored (reperfusion damage). In studies of cardiac patients, deficiencies of the enzyme were found in 75% of 132 biopsy specimens of heart tissues, and 20% of 406 blood samples. Studies performed by several different groups of researchers have shown that supplementation with Coenzyme Q₁₀ improves the signs and symptoms of CAD at doses of 1.5 mg/kg per day (90 mg in a 60 kg person), 150 mg/day and 600 mg/day. Greenberg & Frishman, J. Clinical Pharmacology 30: 596-608 (1990) at p. 599. Earlier clinical studies in Japan used a dose of 5 mg, and later a dose range of 25-100 mg. Folkers, et al., J. Molecular Medicine, 2:431-460 (1977).

U.S. Pat. No. 6,576,242 discloses that administering a composition including red yeast rice in combinations with coenzyme Q₁₀ and tocopherols is effective for reducing or controlling blood triglycerides and thus is a useful as a treatment of artherosclerosis. The compositions of the present invention optionally include at least one of coenzyme Q₁₀ (for example, at a concentration of about 0.1-10% wt/wt) and a tocopherol, preferably alpha tocopherol (for example, at a concentration of about 0.1-5% wt/wt)/

Coenzyme Q₁₀, mixed tocopherols (vitamin E), selenium, chromium, and inositol hexaphosphate are available commercially, in bulk and wholesale, from suppliers well known to those with ordinary skill in the art. For instance, Vitamin E may be obtained from Ava Health PO Box 730, Grove City, Ohio 43123-0730 and Wholesale Vitamins USA, Inc., of Brooklyn, N.Y. offers over 8,000 vitamins at wholesale prices

As used herein, the tern “effective treatment” means the reduction of a particular symptom, or the significant change of a particular laboratory test toward the normal value, Preferably symptoms are relieved by at least 30-70% and a laboratory test is moved at least 10% toward the normal value; more preferably symptoms are reduced by 70% and/or a laboratory test is moved at least 20% toward the normal value; most preferably, a treatment is effective if the symptoms are reduced by 90%, and/or laboratory parameters are returned to the noirmal value.

The term “hypercholesterolemia” means the presence of elevated levels of cholesterol in the blood.

The term “therapeutically effective amount” or “therapeutic dose” as used herein means the amount of a particular agent sufficient to provide a therapeutic benefit in the treatment or prevention of a disease, or in modulating the level of serum lipids and lipoproteins.

The term “dietary supplement” as used herein means an additional element that is added to the daily food intake of a mammal, usually a human.

Embodiments of the present invention further encompasses a composition comprising a therapeutically effective amount of a product including a pomegranate product and a plant product fermented with a statin-producing fungus, useful for the modulation of serum lipid and lipoprotein levels in a human in need of therapy to maintain the lipid and lipoprotein levels within a healthy normal range. In one embodiment of the invention, the composition is adapted for use in the treatment or prevention of hypertriglyceridemia. In a preferred embodiment, such a composition is used for reducing serum cholesterol and serum triglyceride levels in humans.

As used herein, examples of cardiovascular diseases may include but are not limited to myocardial infarction, coronary heart disease, atherosclerosis, arteriosclerosis. The present invention includes the treatment or prevention of cerebrovascular disease such as stroke, memory loss due to stroke, and cerebral thrombosis.

Unless otherwise defined, all technical and scientific terms used herein have the same meaning as commonly understood by one of ordinary skill in the art to which this invention belongs. Although methods and material similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Any dosage form may be employed for providing the patient with an effective dosage of the composition. Dosage forms include tablets, capsules, dispersions, suspensions, solutions, and capsules etc.

Tablets and capsules represent the most advantageous oral dosage unit form. Any method known to those of ordinary skill in the art may be used to prepare capsules, tablets, or other dosage formulations. Pharmaceutically acceptable carriers include binding agents such as pregelatinized maize starch, polyvinylpryrrolidone or. hydroxypropyl methycellulose; binders or fillers such as lactose, pentosan, microcrystalline cellulose or calcium hydrogen phosphate; lubricants such as magnesium stearate, talc or silica; disintegrants such as potato starch or sodium starch; or wetting agents such as sodium lauryl sulfate. Tablets or capsules can be coated by methods well known to those of ordinary skill in the art.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the active ingredient sufficient to achieve the desired effect (minimal effective concentration, MEC). The MEC will vary for each preparation, but can be estimated from in vitro data. Dosages necessary to achieve the ME-C will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. In prophylactic treatment, administration of doses is generally continued over a prolonged period.

The amount of a composition to be administered will, of course, be dependent on the subject being treated, the severity of the affliction, the manner of administration, the judgment of tile prescribing physician, etc.

Products according to the present invention may be further incorporated into an article of manufacture including instructions for use.

Products of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

According to one aspect of the invention a composition is provided comprising a pharmaceutically acceptable combination of the composition and at least one carrier. Pharmaceutically acceptable carriers for inclusion into the present compositions include carriers most suitable for combination with lipid-based drugs such as diluents, excipients and the like which enhance its oral administration Suitable carriers include, but are not limited to, sugars, starches, cellulose and derivatives thereof, wetting agents, lubricants such as sodium lauryl sulfate, stabilizers, tabletting agents, anti-oxidants, preservatives, coloring agents and flavoring agents Reference may be made to Remington's Pharmaceutical Sciences, (17th ed. 1985) for other carriers that would be suitable for combination with the present compositions. As will be appreciated, the pharmaceutical carriers used to prepare compositions in accordance with the present invention will depend on the administrable form to be used.

In a preferred aspect of the invention, a composition of the present invention is administered to reduce or control blood cholesterol levels in persons having a total cholesterol of 240 mg/DL (5.95 mmol/L) or higher In another embodiment of the invention, the compositions are administered to reduce levels of LDL-cholesterol in persons with an LDL-cholesterol of 130 mg/dL, (3.41 mmol/L) or higher. In yet another embodiment of the invention, the compositions are administered to reduce triglycerides in persons having blood triglycerides of 200 mg/dL (2.26 mmol/L) or higher. In another embodiment, a composition of the present invention is administered to raise levels of HDL to persons with an HDL-cholesterol of 35 mg/dL (1.04 mmol/L) or lower to reduce the risk of atherosclerosis associated with low HDL, levels. The compositions and methods of the present invention may also be utilized to improve or maintain vascular health in specific organ systems including the cardiovascular system, the cereberovascular system, the peripheral vascular system and the intestinal vascular system.

Although throughout this disclosure, a “food supplement” is disclosed, it is noted that the present invention is not limited to embodiments where the supplement is provided as tablets, get caps, or caplets ingested separately as a specific supplement In some embodiments, the food supplement is provided within food products in various forms, i.e., in shake, soup, fruit drink, snack bar and the like. Thus, it is noted that providing the active compounds useful for promoting cardiovascular health within a foodstuff or as a beverage are within the scope of a “food supplement.”

Thus, it is noted that the products of embodiments of the present invention may be combined with any other foodstuff, for example, oils containing the extracts of this invention may be used as cooking oil, frying oil, or salad oil and may be used in any oil-based food, such as margarine, mayonnaise or peanut butter. Grain flour fortified with the compounds of this invention may be used in foodstuffs, such as baked goods, cereals, pastas and soups. Oils containing the extracts and novel anthocyanins extracted therefrom can be emulsified and used in a variety of water-based foodstuffs, such as drinks, including drink mixes as discussed above. Advantageously, such foodstuffs may be included in low fat, low cholesterol or otherwise restricted dietary regimens.

For the purposes of this disclosure, a “nutraceutical” is any functional food that provides an additional benefit other than its nutritional benefit. This category may include nutritional drinks, diet drinks (e.g., Slimfast™., Boost™. and the like) as well as sports herbal and other fortified beverages. The present invention provides nutraceutical compositions that may be used to promote cardiovascular health.

Compositions of the present invention suitable for oral administration may be presented as discrete units such as capsules, cachets, or tablets, each containing a predetermined amount of a product (e.g. pomegranate and fermented plant product), as a powder or granules, or as a solution or a suspension in an aqueous liquid, a nonaqueous liquid, an oil-in-water emulsion, or a water-in-oil liquid emulsion. In general, the compositions are prepared by uniformly and intimately admixing the active ingredient with liquid carriers or finely divided solid carriers or both, and then, if necessary, shaping the product into the desired presentation.

The compositions of the present invention may additionally include binding agents (e.g., pregelatinized maize starch, polyvinylpyrrolidone or hydroxypropyl methylcellulose); binders or fillers (e.g., lactose, pentosan, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets or capsules can be coated by methods well known in the art.

Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats), emulsifying agents (e.g., lecithin or acacia), nonaqueous vehicles (e.g., almond oil, oily esters, ethyl alcohol or fractionated vegetable oils), and preservatives (e.g., methyl or propyl-p-hydroxybenzoates or sorbic acid). The preparations can also be made to resemble foods, containing buffer salts, flavoring, coloring and sweetening agents as appropriate.

Any dosage form may be employed for providing the patient with an effective dosage of the product including pomegranate and fermented plant product. Dosage forms include tablets, capsules, dispersions, suspensions, solutions, capsules and the like. Because of their ease of administration, tablets and capsules represent the most advantageous oral dosage unit form, in which case solid pharmaceutical carriers as described above are employed. In addition to the common dosage forms set out above, the compounds of the present invention may also be administered by controlled release means. However, the most preferred oral solid preparations are capsules.

For example, a tablet may be prepared by compression or molding, optionally, with one more accessory ingredients. Compressed tablets may be prepared by compressing a pomegranate and fermented plant product in a free-flowing form such as powder or granules, optionally mixed with a binder, lubricant, inert diluent, surface active or dispersing agent.

It is noted that in some examples below, the specific embodiments relate to “Rimonest” powder, which is a powder prepared according to any method of US 2005/0118312 of one of the present inventors, incorporated herein by reference. In other examples, the procedure for manufacturing Rimonest powder is modified (i.e. see FIG. 1of US 2005/0118312) so that the seed component ((24) of FIG. 1of US 2005/0118312), and more specifically the seed cake ((21) of FIG. 1of US 2005/0118312) is fermented with a statin producing fungus, and then subsequently dried. The fermented and dried pomegranate seed cake is combined into the slurry ((36) of FIG. 1of US 2005/0118312) as disclosed in US 2005/0118312, and processed substantially identically to the seedcake of US 2005/0118312 to make a modified “Rimonest” powder.

Nonetheless, use of the fermented pomegranate seedcake and “Rimonest” powder is not intended as a limitation of the present invention. Indeed, any pomegranate extract or pomegranate product is within the scope of the present invention. Exemplary pomegranate products include but are not limited to extracts of peels, extracts of seed, fruit or fruit extract, and juice.

For purposes of this specification and the accompanying claims, the terms “pericarp”, “rind” and “peel” are considered synonymous and are used interchangeably.

For purposes of this specification and the accompanying claims, the terms “pericarp extract”, includes an aqueous extract of pomegranate peel.

For purposes of this specification and the accompanying claims, the phrase “seed cake” refers to seeds from which seed oil has been removed by an accepted industrial process. The seeds are preferably, but not necessarily, crushed or ground to increase the yield of seed oil.

For purposes of this specification and the accompanying claims, the phrase “seed oil” includes the result of a process such as, for example, expeller pressing, supercritical fluid extraction with carbon dioxide, solvent extraction and/or lyophilization.

For purposes of this specification and the accompanying claims, the term “juice” refers to unprocessed juice, fermented juice, partially fermented juice, partially dried juice, reduced juice and partially reduced juice.

The present inventors believe that in certain examples, processing the pomegranate products, and employing a synergistic mixture of the pomegranate components, substantially as disclosed in US 2005/0018312, may be useful for increase the therapeutic effects of the presently disclosed compositions and food supplements.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials and steps for carrying out various disclosed functions may take a variety of alternative forms without departing from the invention.

Exemplification

### EXAMPLE 1

### A First Preparation of a Dietary Supplement

A composition of the following formulation is prepared in tablet form by standard methods:

| | |
|---|---|
| Red yeast rice | 225 mg |
| Rimonest Powder | 250 mg |

As used herein, “Rimonest powder” is powder prepared according to the procedure described in paragraphs 65-73 of US 2005/0118312.

Two tablets per day (to be consumed with food) is the recommended dosage for an average weight adult human (70-kg).

### EXAMPLE 2

### A Second Preparation of a Dietary Supplement

A composition of the following formulation is prepared in tablet form by standard methods:

| | | |
|---|---|---|
| Red yeast rice | 225 | mg |
| Rimonest Powder | 250 | mg |
| Coenzyme Q₁₀ | 12 | mg |
| Mixed tocopherols | 100 | IU |

Two tablets per day (to be consumed with food) is the recommended dosage for an average weight adult human (70-log).

### EXAMPLE 3

### A Third Preparation of a Dietary Supplement

A Monascus culture with Rimonest powder is prepared. Preferably this culture includes 5 g Monascus powder per 10 kg of Rimonest powder. The powders are mixed in a mechanical mixer to achieve good distribution. Nutrients, a liquid medium (e.g. water) and yeast are added according to the Fermenter's art.

The relative levels of statins to pomegranate substrate will depend on the fermentation time. The fermentation time is therefore dependent on the desired amount of statins in the fermentation product. Preferred fermentation time is 1-60 days, more preferably 1-50 days, still more preferably 15-40 most preferably 20-30 days.

The fermented product is dried out at preferably 40 degrees under vacuum to form a powder. The dietary supplement is prepared from the powder.

### EXAMPLE 4

### A Fourth Preparation of a Dietary Supplement

This example is carried out like Example 3, except that instead of fermenting Rimonest powder, pomegranate seedcake (i.e. 50-99% defatted) is fermented.

### EXAMPLE 5

### A Fifth Preparation of a Dietary Supplement

According to this example a pomegranate substrate (e.g. Rimonest powder) is fermented with a filamentous fungus and the fermentation product is used in the preparation of a food product. These steps are illustrated below. In general, the fermentation of Rimonest powder with Monascus fungus is similar to fermentation soy powders with Monascus fungus (see U.S. Pat. No. 6,849,281, in particular example sections). The skillful artisan recognizes that certain modification to this process may be made.

Although this example (and other examples) relates to Monascus fungus (e.g. chosen from the group of Monascus ruber), it is appreciated that any statin-producing filamentous fungus is appropriate.

Fermentation is conducted in known way. The fermentation is conducted in at least one fermentation vessel (fermenter) in which a medium comprising Rimonest powder is present as the substrate. Optionally, in some batches, other substrates are added (grains, legumes, etc). The fermentation is started (inoculated) by adding a suspension of spores of the Monascus fungus (inoculum), which has been prepared by fermenting Monascus fungus on a separate medium. The fermentation is executed batch-wise or as a continuous process.

The fermentation involves the following steps, which are executed in the given order:
a) Preparation of the medium for the inoculum and the medium to be used in the fermenter.
b) Sterilization of the media, fermenters and ancillary equipment
c) Production of inoculum
d) Addition of the inoculum to the medium comprising Rimonest powder, for example 5 mg of powder per ml of medium.
e) Conducting the fermentation
f) Removal of the fermentation product from the fermenter

The fermentation product is used in the preparation of the dietary supplement according to the invention.

Optionally, before the fermentation product is used in the preparation of the dietary supplement, the following additional process steps are executed:
g) Sterilization of the fermentation product
h) Drying of the fermentation product (or sterilized fermentation product)

The medium used in tie fermenter is solid or liquid. In case the medium is liquid, usually water is present as a major constituent of the medium.

Care should be taken that the medium contains compounds that can provide a carbon source and a nitrogen source for growth of the Monascus fungus. The medium is sterilized before fermentation, e.g. by heat treatment, i.e. pasteurization.

The medium in the fermenter may contain other substances, which may aid the fermentation, for instance sugars, amino acids and vitamins.

The fermentation may be carried out in a manner, which is determined by the skilled person on the basis of common general knowledge of fermentation technology. By illustration, preferred embodiments are described hereunder.

The fermentation temperature may be important. The temperature is preferably in the range of 10 to 37 degrees C. more preferably 20 to 30 degrees C.

Preferably during fermentation the medium is aerated, e.g. by stirring, shaking etc. Aeration many be carried out by blowing air through the fermentation medium. Preferably the air is wholly or partly saturated with water vapour in case solid state fermentation is used. This avoids drying out of the fermentation medium.

The relative levels of statins to pomegranate substrate will depend on the fermentation time. The fermentation time is therefore dependent on the desired amount of statins in the fermentation product. Preferred fermentation time is 1-60 days, more preferably 1-50 days, still more preferably 15-40 most preferably 20-30 days.

The fermented product is dried out at preferably 40 degrees under vacuum into a powder.

The dietary supplement is formed from the powder.

### EXAMPLE 6

### A Sixth Preparation of a Dietary Supplement

This example is carried out lice Example 5, except that instead of fermenting Rimonest powder, pomegranate seedcake (preferably oil extracted) is fermented.

### EXAMPLE 7

### Trials in Humans—General Information

A study of the effect of eight formulations on various blood parameters (e.g. HDL-cholesterol, LDL-cholesterol, total cholesterol concentrations and plasma triglyceride levels) in the blood of men with elevated cholesterol levels is conducted over a 6 month period.

The formulations are as follows:

FORMULATION A—control placebo tablet of unfermented rice powder.

FORMULATION B—a positive control tablet of Rimonest powder.

FORMULATION C—a positive control tablet of fermented rice yeast rice powder.

FORMULATION D—the formulation of Example 1

FORMULATION E—the formulation of Example 3

FORMULATION F—the formulation of Example 4.

FORMULATION G—the formulation of Example 5

FORMULATION H—the formulation of Example 6

GROUP A is associated with FORMULATION A. GROUP B is associated with FORMULATION B. GROUP C is associated with FORMULATION C. GROUP D is associated with FORMULATION D. GROUP E is associated with FORMULATION E. GROUP F is associated with FORMULATION F. GROUP G is associated with FORMULATION G. GROUP H is associated with FORMULATION H.

There are six groups of patients, each group having no fewer than 10 patients per group.

Thus, a total of eighty men having total plasma cholesterol of between 240 and 300 mg/dL are selected for inclusion in the statistical study. The patients are asked to maintain their diet during this study.

The patients are matched between each group according to age, weight, and other medical conditions, as well as diet. Matching for diet is carried out by ranking the diet of each patient according to its healthfulness from the point of view of preventive medicine and good cardiovascular health. For example, a maximally healthy diet would be one consisting of primarily whole grains, vegetables, legumes and fish. A maximally unhealthy diet would be one consisting mainly of fast foods, excessive cheeses and meats, heavily sugared beverages, etc. The scoring is accomplished via a questionnaire prepared jointly be a dietician/nutritionist and a physician skilled in preventive medicine.

The study is carried out over a 28 week period. For the first 4 weeks, patients from all 6 groups receive FORMULATION A. For the next 20 weeks, patients from each group receive the respective formulation. For the last 4 weeks, patients from each group receive FORMULATION A.

A statistical analysis is performed to compare the resulting levels of certain measured parameters of the patients (determined every four weeks) and each of the three control groups to determine if a significant improvement in levels of certain blood parameters results from administration of the test preparations.

The different measured parameters include total cholesterol, HDL-cholesterol, LDL cholesterol, plasma triglyceride levels, concentrations of serum angiotensin converting enzyme (ACE) activity in the blood, and systolic blood pressure. Each parameter is evaluated using multiple linear regression analysis and a standard Student's t-test. In each analysis the baseline value of the outcome variable is included in the model as a covariant. Treatment by covariant interaction effects is tested by the method outlined by Weigel & Narvaez, Controlled Clinical Trials 12.1 378-94 (1991); If there are no significant interaction effects, the interaction terms are removed from the model. The regression model assumptions of normality and homogeneity of variance of residuals are evaluated by inspection of the plots of residuals versus predicted values. Detection of the temporal outset of effects is done sequentially by testing for the presence of significant treatment effects at the end of each four week period proceeding to the earlier time in sequence only when significant effects have been identified at each later time period. In addition, differences between groups in nutrient intake, physical activity, and body mass index (ht/wt²) at each time point are compared using one-way analysis of variance. Changes from tile baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

### EXAMPLE 8

### Observations—Control Groups B and C

For the patients in Groups B and C, at least one parameter reflecting cardiovascular health (e.g. increase in the ratio of HDL-cholesterol to LDL-cholesterol, a decrease in blood pressure, a decrease in plasma triglyceride levels, a decrease in concentrations of serum angiotensin converting enzyme (ACE) activity in the blood, and reduced systolic blood pressure) is observed to significantly improve relative to Group A.

### EXAMPLE 9

### Observations—Group D

For the patients in Group D, at least one parameter reflecting cardiovascular health (e.g. increase in the ratio of HDL-cholesterol to LDL-cholesterol, a decrease in blood pressure, a decrease in plasma triglyceride levels, a decrease in concentrations of serum angiotensin converting enzyme (ACE) activity in the blood, and reduced systolic blood pressure) is observed to significantly improve relative to Groups A, B and C.

### EXAMPLE 10

### Observations—Group E

For the patients in Group E, at least one parameter reflecting cardiovascular health (e.g. increase in the ratio of HDL-cholesterol to LDL-cholesterol, a decrease in blood pressure, a decrease in plasma triglyceride levels, a decrease in concentrations of serum angiotensin converting enzyme (ACE) activity in the blood, and reduced systolic blood pressure) is observed to significantly improve relative to Groups A, B and C.

### EXAMPLE 11

### Observations—Group F

For the patients in Group F, at least one parameter reflecting cardiovascular health (e.g. increase in the ratio of HDL-cholesterol to LDL-cholesterol, a decrease in blood pressure, a decrease in plasma triglyceride levels, a decrease in concentrations of serum angiotensin converting enzyme (ACE) activity in the blood, and reduced systolic blood pressure) is observed to significantly improve relative to Groups A, B and C.

### EXAMPLE 12

### Observations—Group G

For the patients in Group G, at least one parameter reflecting cardiovascular health (e.g. increase in the ratio of HDL-cholesterol to LDL-cholesterol, a decrease in blood pressure, a decrease in plasma triglyceride levels, a decrease in concentrations of serum angiotensin converting enzyme (ACE) activity in the blood, and reduced systolic blood pressure) is observed to significantly improve relative to Groups A, B and C.

### EXAMPLE 12

### Observations—Group H

For the patients in Group H, at least one parameter reflecting cardiovascular health (e.g. increase in the ratio of HDL-cholesterol to LDL-cholesterol, a decrease in blood pressure, a decrease in plasma triglyceride levels, a decrease in concentrations of serum angiotensin converting enzyme (ACE) activity in the blood, and reduced systolic blood pressure) is observed to significantly improve relative to Groups A, B and C.

### EXAMPLES 12

### Trials in Hamsters—General Information

A study of the effect of eight formulations on various cardiovascular health parameters in hamsters is conducted over a 2 week period.

The formulations are as follows:

FORMULATION A—negative control placebo tablet of unfermented rice powder.

FORMULATION B—a positive control tablet of Rimonest powder.

FORMULATION C—a positive control tablet of fermented rice yeast rice powder.

FORMULATION D—the formulation of Example 1

FORMULATION E—the formulation of Example 3

FORMULATION F—the formulation of Example 4.

FORMULATION G—the formulation of Example 5.

FORMULATION H—the formulation of Example 6.

The various formulations are added to the standard laboratory hamster chow in a concentration of 10% wtlxvt during the twvo week period.

CONTROL GROUP A1 is associated with FORMULATION A and a cholesterol-free diet.

CONTROL GROUP A2 is associated with FORMULATION A and a diet enriched with cholesterol (0 5%, w/w).

CONTROL GROUP B1 is associated with FORMULATION B and a cholesterol-free diet.

CONTROL GROUP B2 is associated with FORMULATION B and a diet enriched with cholesterol (0.5%, w/w).

CONTROL GROUP C1 is associated with FORMULATION C and a cholesterol-free diet.

CONTROL GROUP C2 is associated with FORMULATION C and a diet enriched with cholesterol (0.5%, w/w).

GROUP D1 is associated with FORMULATION D and a cholesterol-free diet.

GROUP D2 is associated with FORMULATION D and a diet enriched with cholesterol (0.5%, v/w).

GROUP E1 is associated with FORMULATION E and a cholesterol-free diet.

GROUP E2 is associated with FORMULATION E and a diet enriched with cholesterol (0.5%, w/v).

GROUP F1 is associated with FORMULATION F and a cholesterol-free diet.

GROUP F2 is associated with FORMULATION F and a diet enriched with cholesterol (0.5%, v/v).

GROUP G1 is associated with FORMULATION G and a cholesterol-free diet.

GROUP G2 is associated with FORMULATION G and a diet enriched with cholesterol (0.5%, w/w).

GROUP H1 is associated with FORMULATION H and a cholesterol-free diet.

GROUP H2 is associated with FORMULATION H and a diet enriched with cholesterol (0.5%, w/w).

There are sixteen groups of hamsters, each group having no fewer than 10 hamsters per group.

The following parameters reflecting cardiovascular health are measured after the two week trial:

BLOOD SERUM PARAMETERS: HDL-cholesterol, LDL-cholesterol, total cholesterol concentrations and plasma triglyceride levels)

In addition, animals are sacrificed, and liver triglyceride concentrations are measured.

### EXAMPLE 13

### Observations—Hamster Control Group A

In control Group A, cholesterol feeding induces hyper triglyceridedemia and hypercholesterolemia in the animals. Moreover, liver triglyceride concentrations increase in the animals fed the cholesterol enriched diet.

### EXAMPLE 14

### Observations—Hamster Control Groups B-C

Modest improvements in one or more parameters (i.e. HDL-cholesterol, LDL-cholesterol, total cholesterol concentrations and plasma triglyceride levels) are observed for Formulations B and C relative to Formulation A. In addition, liver triglyceride concentrations are reduced in the animals of Group B-C relative to the animals of Groups A. These improvements are more pronounced in the groups fed a cholesterol enriched diet.

### EXAMPLE 15

### Observations—Hamster Group D

Modest improvements in one or more parameters (i.e. HDL-cholesterol, LDL-cholesterol, total cholesterol concentrations and plasma triglyceride levels) are observed for Group D relative to Groups A-C. This improvement is more pronounced in the groups fed a cholesterol enriched diet.

In addition, liver triglyceride concentrations are reduced in the animals of Group D relative to the animals of Groups A-C. This suggests that when we the combined product is used, there is a supra-additive effect indicative of synergy.

### EXAMPLE 16

### Observations—Hamster Group E

Modest improvements in one or more parameters (i.e. HDL-cholesterol, LDL-cholesterol, total cholesterol concentrations and plasma triglyceride levels) are observed for Group E relative to Groups A-C. This improvement is more pronounced in the groups fed a cholesterol enriched diet.

In addition, liver triglyceride concentrations are reduced in the animals of Group E relative to the animals of Groups A-C. This suggests that when we the combined product is used, there is a supra-additive effect suggesting a synergistic effect.

### EXAMPLE 17

### Observations—Hamster Group F

Modest improvements in one or more parameters (i.e. HDL-cholesterol, LDL-cholesterol, total cholesterol concentrations and plasma triglyceride levels) are observed for Group F relative to Groups A-C. This improvement is more pronounced in the groups fed a cholesterol enriched diet.

In addition, liver triglyceride concentrations are reduced in the animals of Group F relative to the animals of Groups A-C. This suggests that when we the combined product is used, there is a supra-additive effect indicative of synergy.

### EXAMPLE 18

### Observations—Hamster Group G

Modest improvements in one or more parameters (i.e. HDL-cholesterol, LDL-cholesterol, total cholesterol concentrations and plasma triglyceride levels) are observed for Group G relative to Groups A-C. This improvement is more pronounced in the groups fed a cholesterol enriched diet.

In addition, liver triglyceride concentrations are reduced in the animals of Group G relative to the animals of Groups A-C. This suggests that when we the combined product is used, there is a supra-additive effect indicative of synergy.

### EXAMPLE 19

### Observations—Hamster Group H

Modest improvements in one or more parameters (i.e. HDL-cholesterol, LDL-cholesterol, total cholesterol concentrations and plasma triglyceride levels) are observed for Group H relative to Groups A-C. This improvement is more pronounced in the groups fed a cholesterol enriched diet.

In addition, liver triglyceride concentrations are reduced in the animals of Group F relative to the animals of Groups A-C This suggests that when we the combined product is used, there is a supra-additive effect indicative of synergy.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

What is claimed is:

1) A dietary supplement useful for treating or preventing cardiac disease, the supplement comprising a pomegranate product fermented with a statin-producing fungus.

2) The supplement of claim 1, wherein said pomegranate product includes a pomegranate seed component, and said pomegranate seed component includes at least one item selected from the group consisting of a seed cake, seeds, milled seeds and seed powder.

3) The supplement of claim 2 wherein said seed cake is fermented with said statin-producing fungus.

4) The method of claim 3 further comprising a plant product other than said pomegranate product fermented with a said statin-producing fungus.

5) The supplement of claim 1 wherein said pomegranate product includes a pomegranate seed component, and said pomegranate seed is at least partially oil extracted.

6) The supplement of claim 1 further comprising at least one of coenzyme Q₁₀ and a tocopherol.

7) An article of manufacture, the article of manufacture comprising the product of claim 1, packaging material and instructions for use identifying said product as useful for at least one of correcting or preventing a blood lipid dyscrasia and lowering blood pressure in a hypertensive patient.

9) An article of manufacture, the article of manufacture comprising the product of claim 1, packaging material and instructions for use, wherein said product is supplied in an orally administrable form selected from the group consisting of consisting of a tablet and a capsule.

10) A method of correcting or preventing a blood lipid dyscrasia in a subject, the method comprising administering to a patient the supplement of claim 1.

11) A method of lowering blood pressure in a hypertensive patient in a subject, the method comprising administering to a patient the supplement of claim 1.

12) A dietary supplement useful for treating or preventing cardiac disease, the supplement comprising:
a) a pomegranate product; and
b) a plant product fermented with a statin-producing fungus.

13) The mixture of claim 12 wherein said plant product includes a grain product.

14) The mixture of claim 12 wherein said grain product is selected from the group consisting of barley, wheat, rice, corn, oats, sphelt, buckwheat, rye.

15) The mixture of claim 12 wherein said plant product includes soybeans.

16) The supplement of claim 12 further comprising at least one of coenzymeQ₁₀ and a tocopherol.

17) An article of manufacture, the article of manufacture comprising the product of claim 12, packaging material and instructions for use identifying said product as useful for at least one of correcting or preventing a blood lipid dyscrasia and lowering blood pressure in a hypertensive patient.

18) An article of manufacture, the article of manufacture comprising the product of claim 12, packaging material and instructions for use, wherein said product is supplied in an orally administrable form selected from the group consisting of consisting of a tablet and a capsule.

19) A method of correcting or preventing a blood lipid dyscrasia in a subject, the method comprising administering to a patient the supplement of claim 12.

20) A method of lowering blood pressure in a hypertensive patient in a subject, the method comprising administering to a patient the supplement of claim 12.

21) A method of preparing a dietary supplement useful for treating or preventing cardiac disease, the method comprising:
a) providing a pomegranate product;
b) fermenting said pomegranate product with a statin-producing fungus.

22) A method of preparing a dietary supplement useful for treating or preventing cardiac disease, the method comprising:
a) providing a pomegranate product;
b) providing a plant product fermented with a statin-producing fungus; and
c) mixing said pomegranate product and said plant product to form the dietary supplement.
